(19) United States
(12) Patent Application Publication
(10) Pub. No.: US 2015/0125342 A1
(43) Pub. Date: May 7, 2015

(54) Controlled Retention and Removal of Biomaterials and Microbes

(71) Applicant: Kimberly-Clark Worldwide, Inc., Neenah, WI (US)

(72) Inventors: Jose K. Abraham, Neenah, WI (US); Kaiyuan Yang, Cumming, GA (US); David W. Koenig, Menasha, WI (US); Joseph R. Feldkamp, Appleton, WI (US)

(73) Assignee: Kimberly-Clark Worldwide, Inc., Neenah, WI (US)

(21) Appl. No.: 14/072,833

(22) Filed: Nov. 6, 2013

(51) Int. Cl. A61L 2/03

(52) U.S. Cl. CPC A61L 2/03
USPC 422/22; 422/186.05

(57) ABSTRACT

A system for removing microbes from a surface, where the microbes are retained by a film, or a film that can prevent microbes from attaching on a surface are described, where the film is electrically connected to a voltage source via surface electrodes. The film can include a tunable dielectric material, and the dielectric constant of the dielectric material can be adjusted to alter the attachment of microbes on the surface when the surface is contacted by the dielectric material. The surface to be contacted can include any surface present in households, water treatment facilities, food industry facilities, soil remediation, or medical facilities. Such surfaces can include tables, countertops, walls, cabinets, doors, door handles, door knobs, etc. The system can also be used to treat any devices used in the aforementioned environments, such as food preparation equipment, medical devices, water cooler tower equipment, etc.

### BACKGROUND

Biofilms are aggregates of microorganisms where cells adhere to each other on a surface. The cells are often embedded within a self-produced matrix of extracellular polymeric substance (EPS). The EPS is a polymeric conglomeration that generally contains extracellular lipids, proteins, and polysaccharides. Formation of a biofilm begins with the attachment of free-floating microbes to a surface. The microbes adhere to the surface initially through weak, reversible van der Waals forces. However, if the microbes are not immediately separated from the surface, they can anchor themselves more permanently using cell adhesion appendages such as pili. Once microbe colonization has begun, the biofilm grows through a combination of cell division and recruitment.

Biofilms can be found on any surface such as solid substrates submerged in or exposed to some aqueous solution or other microbe-containing media. Microbes such as bacteria, fungi, and viruses living in a biofilm can have significantly different properties from free-floating microbes, as the dense and protected environment of the film allows them to cooperate and interact in various ways. This results in increased resistance to detergents, biocides, and antibiotics, as the dense extracellular matrix and the outer layer of cells protect the interior of the community.

Biofilms can form on living or non-living surfaces and can exist in natural and industrial settings. For instance, biofilms can contaminate man-made aquatic systems such as cooling towers, medical lines, medical devices, spas, etc. In industrial environments, biofilms can develop on the interiors of pipes and lead to clogs and corrosion. In medicine, biofilms spreading along implanted tubes or wires can lead to infections in patients. Further, biofilms on floors and counters can make sanitation difficult in food preparation areas. Due to these detrimental effects, various means for controlling or dispersing biofilms have been developed.

Currently, biofilms can be removed through the use of chemical solutions containing antibiotics and biocides. However, these treatments often require high concentrations of potentially toxic chemicals, raising environmental concerns, and these treatments are often very expensive. Further, there is concern antimicrobial resistance (AMR) caused by overuse of antimicrobial treatments, AMR results in resistant organisms (including bacteria, fungi, viruses and some parasites) being able to withstand attack by antimicrobial medicines, such as antibiotics, antifungals, antivirals, and antimalarials, so that standard treatments become ineffective and infections persist increasing risk of spread to others. The evolution of resistant strains is a natural phenomenon that happens when microorganisms are exposed to antimicrobial drugs, and resistant traits can be exchanged between certain types of bacteria. The misuse of antimicrobial medicines accelerates this natural phenomenon.

In light of the above, a need exists for a system and method of disrupting and then retaining microbes from a biofilm to remove the biofilm from any desired surface without the use of antibiotics, biocides, or other chemical treatments. A method and system of treating a surface such that biofilm formation is prevented would be particularly useful.

### SUMMARY

Aspects and advantages of the invention will be set forth in part in the following description, or may be obvious from the description, or may be learned through practice of the invention.

One exemplary aspect of the present disclosure is directed to a system for altering the attachment of microbes to a surface. The system can include a film that can be a tunable dielectric material; a plurality of electrodes positioned on an outer surface of the film; a voltage source electrically connected to the plurality of electrodes; and a control circuit configured to control application of a voltage from the voltage source to the tunable dielectric material to alter the attachment of microbes to the surface.

In one embodiment, the tunable dielectric material can have a first dielectric constant ranging from about 3 to about 10,000 when no voltage has been applied. Further, the first dielectric constant can change by an amount of from about 1% to about 80% to result in a second dielectric constant when a voltage is applied.

In one particular embodiment, the film can be a composite comprising the tunable dielectric material and an additional material, wherein the additional material can include a polymer, a ceramic, or a combination thereof. The tunable dielectric material can be present in an amount ranging from about 5 wt. % to about 99 wt % and the additional material can be present in an amount ranging from about 1 wt. % to about 50 wt. % based on the total weight of the substrate. It should be understood that the additional material can be part of a composite with the dielectric material, or the additional material can be added as an application layer on a selective surface.

In another embodiment, the tunable dielectric material can be a ferroelectric material comprising barium titanate, barium strontium titanate, lead titanate, or a combination thereof.

In still another embodiment, the plurality of electrodes can be arranged in an interdigitated pattern. In another embodiment, the system can further include a display.

In yet another embodiment, the film can be disposed on a substrate.

In one embodiment, the system can enhance the attachment of microbes to the surface. Meanwhile, in another embodiment, the system can prevent the attachment of microbes to the surface.

Another exemplary aspect of the present disclosure is directed to a method for the altering the attachment of microbes to a surface. The method can include contacting the surface with a film, the film comprising a tunable dielectric material; and applying a voltage to a plurality of electrodes positioned on the film to tune a dielectric constant of the tunable dielectric material from a first dielectric constant to a second dielectric constant, whereby tuning the tunable dielectric material can alter the attachment of microbes to the film.

In one embodiment, the second dielectric constant can exhibit a change of from about 1% to about 80% compared to the first dielectric constant. Further, the voltage applied can range from about 1 volt to about 100 volts. It should be understood, however, that the applied voltage for implantable devices should be less than 50 volts, while the applied voltage for surface cleaning applications can be greater than 100 volts.

In still another embodiment, the voltage can comprise a DC component and an AC component. The DC component can tune the dielectric constant of the tunable dielectric material. Meanwhile, the AC component can be applied at a frequency ranging from about 1 hertz to about 20 megahertz. Further, the strength of a resulting electric field can range from about 0.01 volts/micrometer to about 40 volts/micrometer.

In an additional embodiment, tuning the dielectric material can reduce the attachment of microbes to the film. In such an embodiment, the film can be part of an implantable medical device where the film can be coated onto the surface. Meanwhile, in still another embodiment, tuning the tunable dielectric material can enhance the attachment of microbes to the film. In such an embodiment, the film can be part of a system for cleaning the surface.

In another embodiment, the tunable dielectric material can include a ferroelectric material that comprises barium titanate, barium strontium titanate, lead titanate, or a combination thereof.

These and other features, aspects and advantages of the present invention will become better understood with reference to the following description and appended claims. The accompanying drawings, which are incorporated in and constitute a part of this specification, illustrate embodiments of the invention and, together with the description, serve to explain the principles of the invention.

### BRIEF DESCRIPTION OF THE DRAWINGS

A full and enabling disclosure of the present invention, including the best mode thereof to one skilled in the art, is set forth more particularly in the remainder of the specification, including reference to the accompanying figures, in which:

FIG. 1 is a perspective view of a biofilm cleaning system according to an exemplary embodiment of the present disclosure;

FIG. 2 is a flow chart for a method of controlling the applied voltage of a film containing a ferroelectric material to remove microbes from a surface or adhere microbes to a surface according to an exemplary embodiment of the present disclosure;

FIG. 3 is a top view of a substrate that includes a dielectric material in the form of a film as well as first and second electrodes according to an exemplary embodiment of the present disclosure;

FIG. 4 is a cross-sectional view of an implantable medical device treated with a substrate for preventing formation of a biofilm according to an exemplary embodiment of the present disclosure;

FIG. 5 illustrates the change in electric field distribution inside a tunable dielectric material for the four cases described in Table 1; and

FIG. 6 illustrates the change in dielectric constant based on the cases described in Table 1.

Repeat use of reference characters in the present specification and drawings is intended to represent the same or analogous features or elements of the present invention.

### DETAILED DESCRIPTION

It is to be understood by one of ordinary skill in the art that the present discussion is a description of exemplary embodiments only, and is not intended as limiting the broader aspects of the present disclosure.

Generally speaking, the present disclosure is directed to a method and system for removing biofilm microbes from a surface or preventing the attachment of microbes to a surface. The biofilm prevention and removal system can be used to remove microbes, including bacteria, fungi, and viruses, from any surface to which a biofilm can adhere or attach, such as any surfaces present in households, water treatment facilities, food industry facilities, soil remediation, or medical facilities. Such surfaces can include tables, countertops, walls, cabinets, doors, door handles, door knobs, etc. The system can also be used to treat any devices used in the aforementioned environments, such as food preparation equipment, medical equipment and devices, water cooler tower equipment, etc. In one embodiment, the system can include a substrate (base material) to which a tunable dielectric material in the form of a thin film has been attached or adhered, surface electrodes attached to the film, a voltage source, a control circuit, and a display. The voltage source can be configured to provide voltage to the electrodes. The voltage can have a direct current (DC) component and an alternating current (AC) component.

In another embodiment, the present disclosure contemplates a medical device coated with tunable dielectric material in the form of a film on which surface electrodes have been attached. Such coating can limit biofilm formation by preventing the attachment of microbes, including bacteria, fungi, and viruses to a surface. Instead of taking the form of a coating, it should also be understood that the tunable dielectric material can be adhered to the medical device and can be supported by a substrate (base material) as discussed above. In such an embodiment, the dielectric constant of the tunable dielectric material is not altered until after implantation of the medical device into the body. It is also to be understood that the coating or film containing the tunable dielectric material can also be used to prevent scar tissue formation around a medical device after implantation.

Turning first to the tunable dielectric material (e.g., an outermost functional layer of a multi-layered structure), in some embodiments, the material can be in the form of a thin film that includes a tunable dielectric material. The film can have a thickness ranging from about 0.001 micrometers to about 10 millimeters, such as from about 5 micrometers to about 5 millimeters, such as from about 50 micrometers to about 3 millimeters. In one embodiment, the film can be a molecular monolayer having a thickness ranging from about 0.001 to about 5 millimeters. However, it is also to be understood that in another embodiment, the film can be in the form of a coating that is applied to the surface or device on which a biofilm can form via spin coating, vacuum deposition, screen printing, 3D printing, or any other suitable coating method.

Generally, the tunable dielectric material can be any material having an adjustable dielectric constant upon the application of a voltage. For instance, the tunable dielectric material can be a ferroelectric material or a composite material that comprises a ferroelectric material and one or more polymers and/or ceramic materials. An example of a suitable ferroelectric material is a perovskite.

Whether used as part of a surface coating on a device to prevent microbe attachment or in a system to treat a surface by detaching microbes present on the surface and trapping such microbes in the system, the dielectric constant of the tunable dielectric material can be adjusted or tuned to a higher or lower value as desired. For instance, a different dielectric constant is desired when a substrate containing the dielectric material is being used as part of a system to remove microbes from a surface versus when a substrate containing the dielectric material is being sued to prevent attachment of microbes to a surface. For example, when microbes are bathed in a medium having a higher dielectric constant than the tunable dielectric film, microbes show less binding to that film. On the other hand, if the microbes are bathed in a medium of lower dielectric constant than the tunable film, more microbe binding is expected to the tunable dielectric film. As such, the dielectric constant of the dielectric material should be tunable or adjustable such that the dielectric constant can be tuned or adjusted due to the application of voltage as mentioned above. However, it is to be understood that in some embodiments, the application of a magnetic field or vibration can be used to adjust the dielectric constant of the dielectric material.

For instance, in one embodiment, the dielectric material can be tunable such that its dielectric constant can be adjusted via the application of voltage through a voltage source. By adjusting the dielectric constant, the attachment of microbes to a tunable dielectric material or the detachment of microbes from a tunable dielectric material can be selectively controlled.

The voltage applied can range from about 1 volt to about 100 volts, such as from about 1 volt to about 50 volts, such as from about 2 volts to about 40 volts, such as from about 5 volts to about 30 volts. The voltage can include a DC component and an AC component. As a result of the change in the dielectric constant of the tunable dielectric material brought about by the applied voltage, microbes such as gram negative bacteria, gram positive bacteria, fungi, viruses, scar tissue, other biomaterials, etc. can be inhibited from forming into biofilms on a surface coated with the tunable dielectric material. Meanwhile, when a surface on which a biofilm has already been formed is contacted with the dielectric material that has been tuned or adjusted to maintain a high dielectric constant, microbes can be detached from the surface and subsequently attach to electrodes present on the tunable dielectric material due to the selective control of the dielectric constant.

Generally, as a result of the DC component of the applied voltage, the dielectric constant can change by about to about 1% to about 80%, such as from about 5% to about 60%, such as from about 7.5% to about 40%. In one example, for instance, when about 25 volts of DC voltage are applied, the dielectric constant can change by about 30%.

Further, the AC component of the applied voltage can have a specified range of frequencies that can contribute to the antimicrobial effects of the system and method of the present disclosure. For instance, the AC voltage can be applied at a frequency ranging from about 1 hertz (Hz) to about 20 megahertz (MHz), such as from about 2.5 Hz to about 15 MHz, such as from about 5 Hz to about 10 MHz. The AC voltage applied can result in an electric field having a strength ranging from about 0.01 volts per micrometer to about 40 volts per micrometer, such as from about 0.5 volts per micrometer to about 30 volts per micrometer, such as from about 1 volt per micrometer to about 25 volts per micrometer, which field strengths are sufficient to have an antimicrobial effect. For instance, bacterial adhesion can be promoted or reduced by selective implementation of voltages between frequencies ranging from about 1 Hz to about 20 MHz, where it is thought that the dielectrophoretic force strongly influences the polarization effects along with conduction of the microbe cell walls, resulting in biomaterial separation under the influence of the resulting electric field. As the frequency is increased, the electric field is able to penetrate into the cell wall and selective tuning or adjusting of the frequency can be used to separate and remove bacteria from a surface.

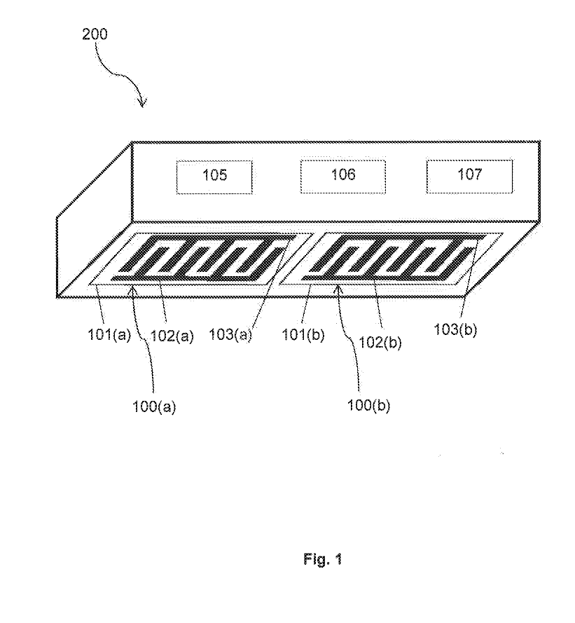

Turning now to FIG. 1, one embodiment of a system 200 for removing microbes from a biofilm attached to a surface is shown. The system can include a voltage source 105 that includes DC and AC components, multiple tunable dielectric materials 100a and 100b each having first electrodes 102a and 102b and second electrodes 103a and 103b disposed thereon. A control circuit 106 can be used to alter whether DC voltage or AC voltage is applied and at what frequency, while a display 107 can show the voltage being applied and at what frequency. Further, it is to be understood that the display can provide other information in addition to the voltage and frequency.

Although only two tunable dielectric material blocks 100a and 100b are shown, it is to be understood that any number of blocks can be utilized depending on the size and shape of the surface to be treated. To treat a surface (not shown), the films 101a and 101b are placed in direct contact with the surface, after which the appropriate voltage can be applied at the appropriate frequency to create an electric field whereby microbes are attracted toward and retained within the dielectric material blocks 100a and 100b, as discussed above in more detail.

Further, the control circuit 106 can include one or more control devices, such as a microcontroller, a microprocessor, an integrated circuit logic device, or any other control device. In one particular embodiment, the control circuit comprises a processor configured to execute computer-readable instructions stored in non-transitory computer readable media to cause the processor to perform operations, such as tuning the tunable dielectric materials 101(a) and 101(b).

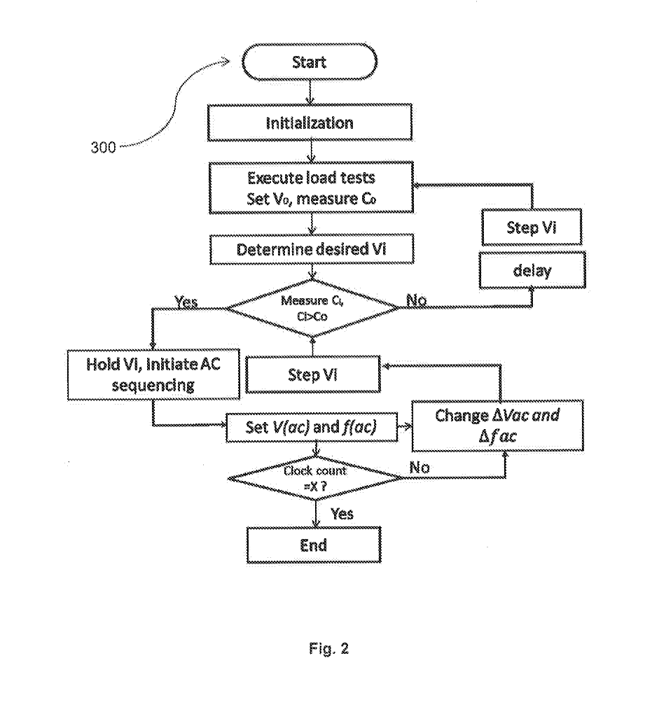

A general flow chart depicting a method 300 used in determining the DC and AC voltages to be applied to a tunable dielectric material, and at what frequency, to effective control microbe adhesion or detachment, is shown in FIG. 2. The sequential controls of the control circuit are initiated upon switching the power supply ON at the start. The circuit then executes necessary load tests from the measured capacitance values (test, Cᵢ, and reference, C₀, values) and decides initial voltage Vᵢ required to change in permittivity of the material. If the measured (test) capacitance, Cᵢ, is greater than the reference capacitance, C₀, the circuit provides constant DC voltage to the electrodes (discussed in more detail below) so that a desired dielectric constant/permittivity can be achieved. The initial reference value of C₀ is selected from a look-up table embedded in a microcontroller. The system holds for a short delay time and changes the step voltage, Vᵢ, if there is no change in Cᵢ from the reference value. The process repeats until it reads the desired Cᵢ by applying more voltage, Vᵢ. Upon measuring desired the Cᵢ, which corresponds with the desired dielectric constant or permittivity, the circuit initiates AC voltage sequencing to effectively control the adhesion or dispersion of microbes or other biomaterials on a surface. The AC voltage and frequency can be controlled to have a minimal effect on the dielectric constant/permittivity values of the tunable dielectric material. The applied voltage and the frequency selectively attract or repel the microbes/biomaterials as desired. The process can be repeated X times to achieve the desired dielectric constant. Desired C₀ and Cᵢ levels are from about 10 picofarads (pF) to 1 about microfarads (pF). For example, C₀ can be 25 pF and Cᵢ can be 300 nanofarads (nF).

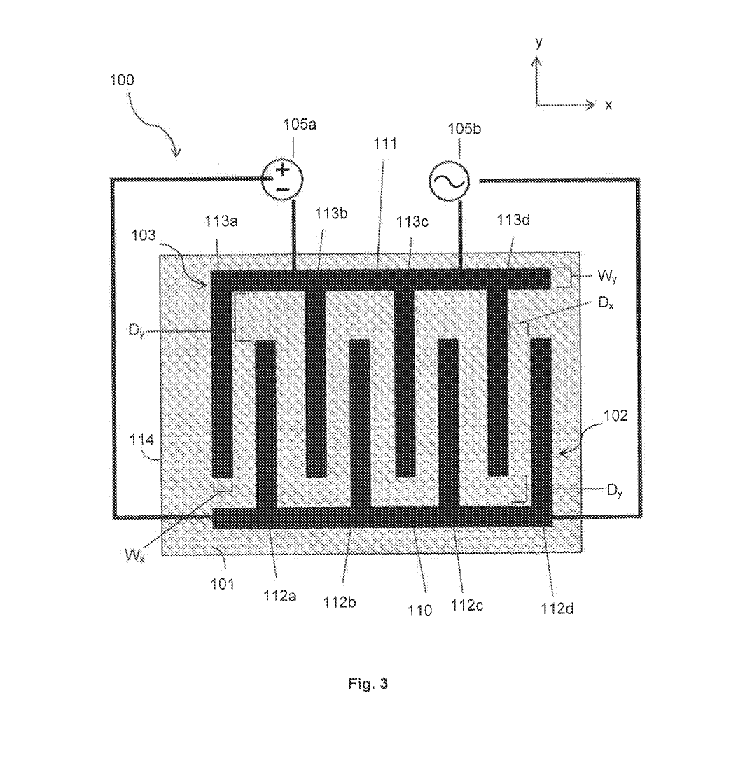

With reference to FIG. 3, an example of one embodiment of a system of the present disclosure will be discussed in greater detail. Generally, a substrate 114 can support the tunable dielectric material 101, which can be a ferroelectric material or a composite that comprises the ferroelectric material and one or more polymers and/or ceramic materials. The ferroelectric material can possess polarization which may be reoriented by the application of an external electric field. Examples of ferroelectric materials include, without limitation, perovskites, tungsten bronzes, bismuth oxide layered materials, pyrochlores, alums, Rochelle salts, dihydrogen phosphates, dihydrogen arsenates, guanidine aluminum sulfate hexahydrate, triglycine sulfate, colemanite, thiourea, iron oxide (Fe₃O₄), and polyvinylidene fluoride (PVDF).

Perovskites are a particularly desirable ferroelectric material due to their ability to form a wide variety of solid solutions from simple binary and ternary solutions to very complex multicomponent solutions, as well as for their ability to have a high dielectric constant that is tunable. Some examples include, but are not limited to barium titanate, barium strontium titanate, lead titanate (e.g., BaTiO₃, BaSrTiO₃, BaₓSr₁₋ₓTiO₃ where x is greater than 0 and less than 1, and Pb(Co_{0.25}Mn_{0.5}W_{0.5})O₃) and numerous other forms of barium titanate, barium strontium titanate, and lead titanate doped with niobium oxide, antimony oxide, and lanthanum oxide, to name a few and by way of illustration only. Another material suitable material is lead mangesium niobate-lead titanate (PMN-PT). The ability to form extensive solid solutions of perovskite-type compounds allows one skilled in the art to systematically alter the electrical properties of the material by formation of a solid solution or addition of a dopant phase. In addition, perovskite-related octahedral structures have a structure similar to that of perovskites, and are likewise exemplary ferroelectric materials, such examples including, but not limited to, lithium niobate (LiNbO₃) and lithium tantalate (LiTaO₃). These materials are intended to be included in the term “perovskites.” Additionally, further examples of ferroelectric materials include bismuth oxide layered materials which comprise complex layered structures of perovskite layers interleaved with bismuth oxide layers. An exemplary bismuth oxide layered compound is lead bismuth niobate (PbBiNb₂O₉). A more detailed description of suitable ferroelectric materials is provided in U.S. Pat. No. 6,162,535 to Turkevich et al., the entire contents of which are hereby incorporated herein by reference.

Meanwhile, the polymer in the composite can include polypropylene, polyamide, polyimide, polyetherimide, fluropolymer, polyamide-imide, polyetherketone, polyetherketoneketone, polysulfone, polyphenylene sulfide, polyester, phenolic resin, bismaleide resin, polybutadiene, polyphenylene oxide, epoxy, polyacrylate, polyamide, PVDF, or any other suitable polymer. The ceramic material can include kaolinite, alumina (Al₂O₃), silicon carbide, tungsten carbide, or any other suitable ceramic material.

In some embodiments, the tunable dielectric material can be a film that contains 100 wt. % of ferroelectric material. On the other hand, the amount of ferroelectric material contained in a film that is a composite can range from about 5 wt. % to about 99 wt. %, such as from about 15 wt. % to about 90 wt. %, such as from about 20 wt. % to about 80 wt. % based on the total weight of the film. Meanwhile, the polymer or ceramic material in the composite film can be present in an amount ranging from about 1 wt. % to about 50 wt. %, such as from about 5 wt. % to about 40 wt. %, such as from about 10 wt. % to about 30 wt. % based on the total weight of the substrate. Further, it is also to be understood that the polymer or ceramic material may be present as a coating or base material on which the ferroelectric material is applied or attached rather than as a composite with the ferroelectric material.

In one embodiment, for instance, the tunable dielectric material can comprise a composite made of a polymeric matrix with the ferroelectric material dispersed therein. The ferroelectric material can be located randomly throughout the polymeric matrix and, desirably, is substantially uniformly distributed throughout the polymeric matrix of the particular layer. In this regard, the composite desirably comprises a zero/three composite. As used herein a “zero/three” composite refers to the dimensional connectivity of the ferroelectric material and the polymer comprising the composite. Connectivity is a macroscopic measure of the composite structure which considers the individual structures (i.e. the ferroelectric material and the polymer) continuity in the x, y, and z dimensions. The first number refers to continuity of the ferroelectric material within the composite and a zero rating indicates that the ferroelectric particles form discrete phases which are discontinuous in the x, y and z dimensions. The second number refers to the continuity of the polymeric portion of the composite and a three rating indicates that the polymeric portion of the composite is continuous in each of the x, y and z dimensions.

In addition, the desired particle size of the ferroelectric material can vary with respect to the particular manufacturing process as well as the desired physical attributes of the substrate or composite substrate made therefrom. In one embodiment, the ferroelectric material can have a longest dimension in a range of from about 10 nanometers to about 10 micrometers. In another embodiment, the longest dimension of the average ferroelectric particle can be less than about 2 micrometers. In addition, the ferroelectric material can comprise nanosized particles. Suitable ferroelectric materials can be synthesized to form particles of the desired size and/or can be destructured to form particles of the desired size. As used herein, the term “destructured” and variations thereof means a reduction in size of the ferroelectric particles.

The tunable dielectric material, such as a film containing the ferroelectric material, can be formed and processed by various methods.

In one embodiment, a precursor solution of barium strontium titanate (BST) can be prepared from barium 2-ethyl hexanoate, strontium 2-ethyl hexanoate and titanium tetra isopropoxide (TTIP). Methyl alcohol can then be used as a solvent along with acetyl acetonate. The barium precursor can be dissolved in methyl alcohol and can then be refluxed in a reflux condenser at a temperature of about 80° C. for 5 about hours. Strontium 2-ethyl hexanoate was added to this solution and refluxed for about 5 hours to obtain a yellow color solution. Acetylacetonate was added to the solution as a chelating agent, which prevents the precipitation. This solution was stirred and refluxed for about another 3 hours. Separately, a solution of titanium isopropoxide (TTIP) was prepared in 20 milliliters of methyl alcohol. The TTIP solution can be added to the barium strontium solution drop by drop, and, finally, refluxed for 4 hours at 80° C. Water can then be added to the BST solution drop by drop in order to initiate hydrolysis. This solution can then be refluxed for another 6 hours with a vigorous stirring in a nitrogen atmosphere.

In this particular embodiment, a platinum/silicon substrate was used for the deposition of the BST thin films. The substrates were immersed in the methanol and dried by nitrogen gas to remove the dust particles. The precursor solution was coated on the substrate by spin coating. The spin coating was done using a spinner rotated at a rate of about 3100 rpm for about 30 seconds. After coating onto the substrate, the films can be kept on a hot plate for about 15 minutes to dry and pyrolize the organics. This process can be repeated to produce multilayered films to achieve the desired film thickness. For instance, up to 3, 5, 7, or 11 layers or more can be formed with the repetition of mild heat treatment for about 15 minutes. The resulting films can then be annealed at 150 to 700° C. for 1 hour in an air atmosphere. Although platinum/silicon substrate is described, it is to be understood that any suitable substrate polymer or nonwoven materials or other material can be utilized, such as a ceramic material, polycrystalline silicon, polyimide film, KAPTON® available from DuPont, etc.

In another embodiment, the dielectric material can be a barium strontium titanate film that is deposited onto a substrate via RF sputtering. The BST can be deposited onto the substrate under 10 milliTorr pressure, 10% oxygen gas, and 120 watt RF power at 700° C. After sputtering, the BST films can then be annealed at 700° C. in excess oxygen gas for about 1 hour and cooled.

In another embodiment, when the tunable dielectric material is a composite film, which may or may not require attachment to a substrate as discussed above, the film can be formed by the following process: (i) destructuring the ferroelectric material in the presence of a liquid and a surfactant to give destructured particles, wherein the liquid is a solvent for the surfactant and the surfactant is chosen to stabilize the destructured particles against agglomeration; (ii) forming a composite of the stabilized, destructured ferroelectric material particles and the polymeric component(s) of the layer; and (iii) extruding the composite material to form the layer as desired. A mixture of the stabilized, destructured ferroelectric material particles and a thermoplastic polymer may be prepared by a variety of methods. As specific examples, methods of making such materials are described in U.S. Pat. No. 5,800,866 to Myers et al., the entire contents of which are hereby incorporated herein by reference.

Regardless of the particular ferroelectric material utilized in the tunable dielectric material, the tunable dielectric material can have a high dielectric constant, such as a dielectric constant ranging from about 3 to about 2000 or greater, such as from about 25 to about 500, such as from about 50 to about 300, such as from about 100 to about 200. In any event, the dielectric constant of the material used for the substrate should be tuned to be greater than the dielectric constant of the biofilm to be treated with the system of the present disclosure to facilitate removal of microbes from the surface on which the biofilm has adhered. On the other hand, in another embodiment, the tunable dielectric material of the present disclosure can be used in a substrate that coats a surface to prevent biofilm formation on the surface in the first instance by adjusting or changing the dielectric constant of the surface, where a biofilm is less likely to adhere to the surface when the dielectric constant of the substrate is reduced to have a lower dielectric constant, such as from about 1 to about 10, such as from about 1 to about 8, such as from about 1 to 5. Generally, the tunable dielectric material of the present disclosure can be tuned via the application of DC voltage such that the dielectric constant of the substrate can be adjusted by a percent ranging from about 1% to about 80%, such as from about 5% to about 60%, such as from about 7.5% to about 40% when a DC voltage is applied versus when no DC voltage is applied.

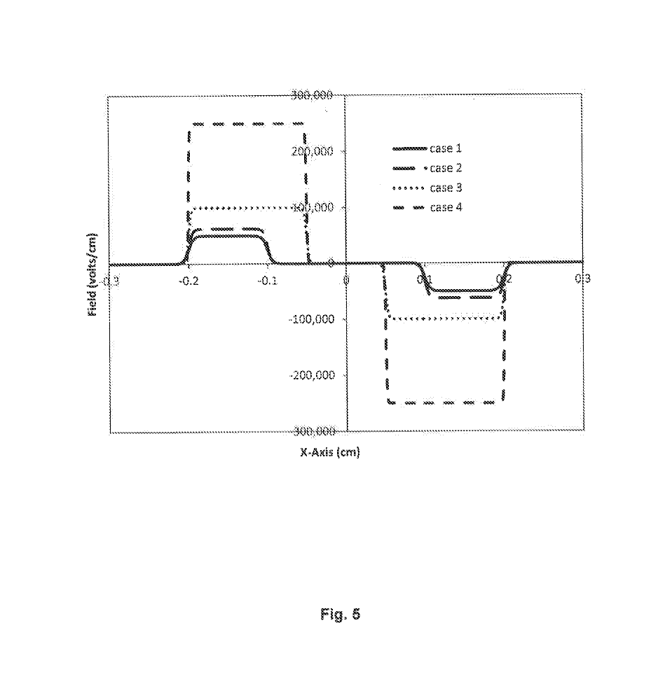

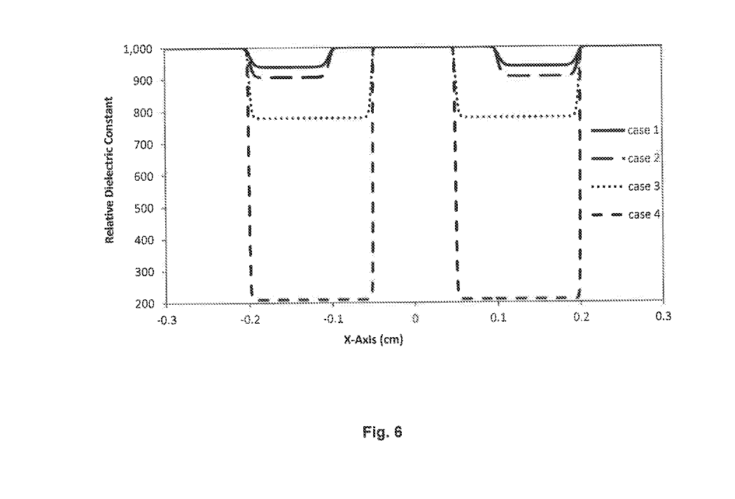

Table 1 below shows the effect of film thickness, electrode width (W_{y} or Wₓ), the distance between electrodes (Dₓ), and the applied voltage on the change in dielectric constant of a tunable dielectric film having an initial dielectric constant of 1000 for four different cases. Meanwhile, FIG. 5 illustrates the change in electric field distribution inside the tunable dielectric film for the four cases of Table 1, while FIG. 6 illustrates the change in dielectric constant for the four cases of Table 1.

TABLE 1

| | Film | Width, | Gap, | Voltage | |
|---|---|---|---|---|---|
| | thickness, | Wy | Dx | (volts/ | Change in dielectric |
| Case: | (μm) | (mm) | (mm) | micrometer) | constant |
| 1 | 100 | 1 | 2 | 0-5 | 1000 to 939 (−6.1%) |
| 2 | 80 | 1 | 2 | 0-6.5 | 1000 to 906 (−9.4%) |
| 3 | 50 | 1.5 | 1 | 0-10 | 1000 to 778 (−22.2%) |
| 4 | 20 | 1.5 | 1 | 0-25 | 1000 to 209 (−79.1%) |

As shown in Table 1 above, if the electric field is varied from 0 volts/micrometer to about 5 volts/micrometer, the dielectric constant can change from 1000 to 939, which is a percent decrease of about 6.1%. Further, if the electric field is varied from 0 volts/micrometer to about 6.5 volts/micrometer, the dielectric constant can change from 1000 to 906, which is a percent decrease of about 9.4%. Meanwhile, if the electric field is varied from 0 volts/micrometer to 10 volts/micrometer, the dielectric constant can change from 1000 to 778, which is a percent decrease of about 22.2%. Lastly, if the electric field is varied from 0 volts/micrometer to 25 volts/micrometer, the dielectric constant can change from 1000 to 209, which is a decrease of about 79.1%. Thus, subjecting the film to a stronger electric field results in a greater change in the dielectric constant.

Meanwhile, the electrodes present on a surface of the tunable dielectric material can typically be formed from a thin film of conductive material disposed on the substrate. Generally speaking, a variety of conductive materials may be used to form the electrodes. Suitable materials include, for example, carbon, metals (e.g., platinum, palladium, gold, tungsten, titanium, etc.), metal-based compounds (e.g., oxides, chlorides, etc.), metal alloys, conductive polymers, combinations thereof, and so forth. Particular examples of carbon electrodes include glassy carbon, graphite, mesoporous carbon, nanocarbon tubes, fullerenes, etc. Thin films of these materials may be formed by a variety of methods including, for example, sputtering, reactive sputtering, physical vapor deposition, plasma deposition, chemical vapor deposition (CVD), printing, spraying, and other coating methods. For instance, carbon or metal paste based conductive materials are typically formed using screen printing, which either may be done manually or automatically. Likewise, metal-based electrodes are typically formed using standard sputtering or CVD techniques, or by electrochemical plating.

Discrete conductive elements may be deposited to form the electrodes, for example, using a patterned mask. Alternatively, a continuous conductive film may be applied to the substrate and then the electrodes may be patterned from the film, Patterning techniques for thin films of metal and other materials are well known in the art and include photolithographic techniques. An exemplary technique includes depositing the thin film of conductive material and then depositing a layer of a photoresist over the thin film. Typical photoresists are chemicals, such as organic compounds, that are altered by exposure to light of a particular wavelength or range of wavelengths. Exposure to light makes the photoresist either more or less susceptible to removal by chemical agents. After the layer of photoresist is applied, it is exposed to light, or other electromagnetic radiation, through a mask. Alternatively, the photoresist is patterned under a beam of charged particles, such as electrons. The mask may be a positive or negative mask depending on the nature of the photoresist. The mask includes the desired pattern of electrodes. Once exposed, the portions of the photoresist and the thin film between the electrodes is selectively removed using, for example, standard etching techniques (dry or wet), to leave the isolated electrode.

The electrodes may have a variety of shapes, including, for example, interdigitated, square, rectangular, circular, ovoid, and so forth. In one embodiment, the width of the electrodes may be from about 0.5 micrometers to about 15 micrometers, in some embodiments from about 0.75 micrometers to about 10 micrometers, and in some embodiments, from about 1 micrometer to about 5 micrometers. For instance, referring to FIG. 3, the first (positive) and second (negative) electrode bases 111 and 113 may have a width W_{y} in the y-direction ranging from about 0.5 micrometers to about 1500 micrometers, in some embodiments from about 0.75 micrometers to about 1000 micrometers, and in some embodiments, from about 1 micrometer to about 500 micrometers, while the electrode stalks 112a-112d and 113a-113d may have a width Wₓ in the x-direction about 0.5 micrometers to about 1500 micrometers, in some embodiments from about 0.75 micrometers to about 1000 micrometers, and in some embodiments, from about 1 micrometer to about 500 micrometers. Meanwhile, the distance Dₓ between individual stalks of the positive electrodes 112a-d and the negative electrodes 113a-d can range from about 0.5 micrometers to about 1500 micrometers, in some embodiments from about 0.75 micrometers to about 1000 micrometers, and in some embodiments, from about 1 micrometer to about 500 micrometers. Further, the distance D_{y} between the stalks of the positive electrodes 112a-d and the base of the negative electrode 11 or between the stalks of the negative electrodes 113a-d and the base of the positive electrode 110 can range from about 0.5 micrometers to about 1500 micrometers, in some embodiments from about 0.75 micrometers to about 1000 micrometers, and in some embodiments, from about 1 micrometer to about 500 micrometers.

The surface smoothness and layer thickness of the electrodes may be controlled through a combination of a variety of parameters, such as mesh size, mesh angle, and emulsion thickness when using a printing screen. Emulsion thickness may be varied to adjust wet print thickness. The dried thickness may be slightly less than the wet thickness because of the vaporization of solvents. In some embodiments, for instance, the dried thickness of the electrode is less than about 100 microns, in some embodiments less than about 50 microns, in some embodiments less than about 20 microns, in some embodiments less than about 10 microns, and in some embodiments, less than about 1 micron.

FIG. 3 shows a tunable dielectric material 100 in the form of a film 101 containing a dielectric material disposed on a substrate 114. A first electrode 102 and a second electrode 103 can be connected to the tunable dielectric material 100 as discussed above. The first electrode 102 and second electrode 103 can be connected to a voltage source 105 that can supply a DC voltage and an AC voltage at a specified frequency to the electrodes depending on the stage of the decision-making process in the control circuit, as discussed in more detail above. For instance, to change the dielectric constant of the dielectric material 101, a DC voltage can be applied via the source 105 at a voltage as discussed above. Meanwhile, to create an electric field to facilitate the adherence of microbes to the substrate 100 via the first electrode 102 and the second electrode 103, an AC voltage can be applied via the voltage source 105 at a voltage level and frequency range as discussed above. The pattern and arrangement of the first electrode 102 and the second electrode 103 is such that microbes from a biofilm can be trapped on the material 100.

For instance, FIG. 3 shows the first electrode 102 and second electrode 103 in the form of an interdigitated pattern where the first electrode 102 has a base 110 and stalks 112a, 112b, 112c, and 112d. Meanwhile, the second electrode 103 has a base 111 and stalks 113a, 113b, 113c, and 113d. The base of the electrodes can have a width W_{y} as shown and discussed in more detail above, while the stalks of the electrodes can have a width Wₓ as shown and discussed in more detail above. The arrangement of the electrodes is such that the first electrode stalks 112a-d and second electrode stalks 113a-d are alternating where a first stalk is not positioned directly next to another first stalk and is instead positioned next to a second stalk. The range of distances Dₓ between a first stalk 112a-d and a second stalk 112a-d are discussed above, as are the range of distances D_{y} between the first stalks 112a-d and the second base 111 and between the second stalks 113a-d and the first base 110. It should be understood that although an interdigitated pattern of bases and stalks is shown, the first and second electrodes 102 and 103 can be arranged in any suitable pattern to maximize the attachment of microbes to the material 100.

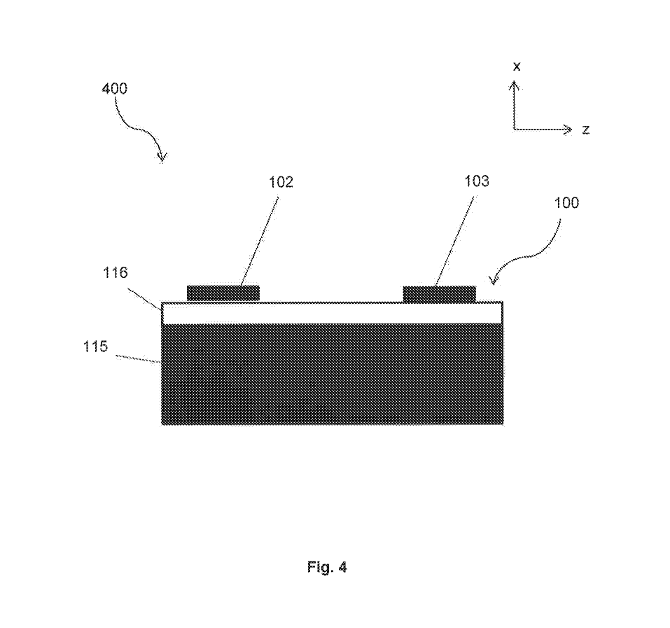

Meanwhile, FIG. 4 shows another embodiment contemplated by the present disclosure. In FIG. 4, a coated medical device 400 is shown. The coated medical device 400 includes medical device 115, to which a tunable dielectric material 100 has been adhered or applied. The material 100 includes a film in the form of a coating 116 containing a tunable dielectric material as well as a positive electrode 102 and a negative electrode 103 disposed on the coating 116, to which AC and DC voltages can be applied to alter the dielectric constant of the tunable dielectric material coating 116. In this manner, through selective control of the dielectric constant of the tunable dielectric material coating 116, microbe attachment to the implantable medical device 115 can be minimized. In this instance, the tuning of the dielectric constant can occur post-implantation.

These and other modifications and variations to the present invention may be practiced by those of ordinary skill in the art, without departing from the spirit and scope of the present invention, which is more particularly set forth in the appended claims. In addition, it should be understood that aspects of the various embodiments may be interchanged both in whole or in part. Furthermore, those of ordinary skill in the art will appreciate that the foregoing description is by way of example only, and is not intended to limit the invention so further described in such appended claims.

What is claimed:

1. A system for altering the attachment of microbes to a surface, the system comprising:
a film, wherein the film comprises a tunable dielectric material;
a plurality of electrodes positioned on an outer surface of the film;
a voltage source electrically connected to the plurality of electrodes; and
a control circuit configured to control application of a voltage from the voltage source to the tunable dielectric material to alter the attachment of microbes to the surface.

2. A system as defined in claim 1, wherein the tunable dielectric material has a first dielectric constant ranging from about 3 to about 10,000 when no voltage has been applied.

3. A system as defined in claim 2, wherein the first dielectric constant changes by an amount of from about 1% to about 80% to result in a second dielectric constant when a voltage is applied.

4. A system as defined in claim 1, wherein the film is a composite comprising the tunable dielectric material and an additional material, wherein the additional material comprises a polymer, a ceramic, or a combination thereof.

5. A system as defined in claim 4, wherein the tunable dielectric material is present in an amount ranging from about 5 wt. % to about 99 wt. % and the additional material is present in an amount ranging from about 1 wt. % to about 50 wt. % based on the total weight of the substrate.

6. A system as defined in claim 1, wherein the tunable dielectric material is a ferroelectric material comprising barium titanate, barium strontium titanate, lead titanate, or a combination thereof.

7. A system as defined in claim 1, wherein the plurality of electrodes are arranged in an interdigitated pattern.

8. A system as defined in claim 1, wherein the film is disposed on a substrate.

9. A system as defined in claim 1, wherein the system enhances the attachment of microbes to the surface.

10. A system as defined in claim 1, wherein the system prevents the attachment of microbes to the surface.

11. A system as defined in claim 1, wherein the system further comprises a display.

12. A method for the altering the attachment of microbes to a surface, the method comprising:
contacting the surface with a film, the film comprising a tunable dielectric material; and
applying a voltage to a plurality of electrodes positioned on the film to tune a dielectric constant of the tunable dielectric material from a first dielectric constant to a second dielectric constant, wherein tuning the tunable dielectric material alters the attachment of microbes to the film.

13. A method as defined in claim 12, wherein the second dielectric constant exhibits a change of from about 1% to about 80% compared to the first dielectric constant.

14. A method as defined in claim 12, wherein the voltage applied ranges from about 1 volt to about 100 volts.

15. A method as defined in claim 12, wherein the voltage comprises a DC component.

16. A method as defined in claim 15, wherein the DC component tunes the dielectric constant of the tunable dielectric material.

17. A method as defined in claim 12, wherein the voltage comprises an AC component.

18. A method as defined in claim 17, wherein the AC component is applied at a frequency ranging from about 1 hertz to about 20 megahertz.

19. A method as defined in claim 18, wherein the strength of a resulting electric field ranges from about 0.01 volts/micrometer to about 40 volts per micrometer.

20. A method as defined in claim 12, wherein tuning the dielectric material reduces the attachment of microbes to the film.

21. A method as defined in claim 20, wherein the film is part of an implantable medical device and the film is coated onto the surface.

22. A method as defined in claim 12, wherein tuning the tunable dielectric material enhances the attachment of microbes to the film.

23. A method as defined in claim 22, wherein the film is part of a system for cleaning the surface.

24. A method as defined in claim 12, wherein the tunable dielectric material comprises a ferroelectric material that comprises barium titanate, barium strontium titanate, lead titanate, or a combination thereof.
